(19) United States
(12) Patent Application Publication
(10) Pub. No.: US 2023/0119743 A1
(43) Pub. Date: Apr. 20, 2023

(54) Hydrophilic Medical Catheters

(71) Applicant: Hao WANG, Chengdu (CN)

(72) Inventor: Hao WANG, Chengdu (CN)

(21) Appl. No.: 17/952,199

(22) Filed: Sep. 23, 2022

(30) Foreign Application Priority Data
Sep. 24, 2021 (CN) 202111125307.0

(51) Int. Cl. A61L 29/08

(52) U.S. Cl. CPC A61L 29/085; A61L 2400/10; A61L 2420/02

(57) ABSTRACT

This invention disclosed medical catheters with surface hydrophilic coatings. Said catheters were grafted with a thin layer of zwitterions, which forms lubricious water layer when contacted with human body liquids or other water solutions, to lower the surface friction and mechanical damage to human body. One benefit of the present invention is due to the excellent biocompatibility and tight bonding between modification material and catheter substrate, the modification will stably stay on the substrate during usage, to avoid the potential side effects caused by lubricants. This modification can be applied to multiple material surfaces, including but not limited to silicone rubber, polyurethane, rubber, polyetheretherketone, polyethylene, polypropylene, polyvinyl chloride, nylon, ABS (Acylonitrile Butadiene Styrene), and polycarbonate.

### FIELD OF INVENTION

The present invention relates to medical catheters, in particular to catheters with a hydrophilic coating on their surface. By reducing the surface friction, the medical catheters are easy to be inserted into patient's body, and the damage caused by mechanical friction on the human body cavity and tissue is hence reduced.

### BACKGROUND

A medical catheter is a medical device commonly used in the clinical practice, which includes urinary catheters, drainage tubes, endotracheal tubes, central venous catheters, rectal tubes, nasogastric feeding tubes and so on. Medical catheters are usually made of polymer materials, such as silicone rubber, polyurethane, natural rubber, polyether ether ketone, polyethylene, polyvinyl chloride, etc. Catheters are inserted into human cavities or tissues to provide a functional channel for the delivery and discharge of gases, liquids, and other components. Due to the frictional resistance with the human cavity or tissue, a catheter is often difficult to insert and even cause damage to the human body. In order to reduce friction, lubricant is sometimes applied to the surface of the catheter, but this increases the discomfort of operation. The lubricating performance is not durable, and the lubricant also increases the risk of blockage and infection. Some catheters, such as intravascular catheters, cannot yet be lubricated. Therefore, to improve the surface properties of catheters by reducing friction and increasing their compatibility with human is a key issue to be solved in the clinical usage of catheters. To achieve this purpose, modifying the surface of the catheter with a hydrophilic coating is the most common method.

U.S. Pat. No. 4,119,094 disclosed a coated substrate having a low coefficient of friction hydrophilic coating and a method of making the same. A substrate is coated with a polyvinylpyrrolidone-polyurethane interpolymer. In the method, a polyisocyanate and a polyurethane in a solvent such as methyl ethyl ketone are applied to a substrate and the solvent evaporated. If the substrate is a polyurethane, only the polyisocyanate need be employed. Polyvinylpyrrolidone in a solvent is then applied to the treated substrate and the solvent evaporated. The invention is applied, for example, to a tube such as a catheter, a catheter and a peristaltic pump tube.

U.S. Pat. No. 6,176,849 disclosed a hydrophilic lubricity coating for medical devices comprising a hydrophobic top coat. The disclosed invention relates to a medical device for insertion into the body wherein the device has at least one surface which periodically comes into contact with a second surface. The first surface comprises an improved lubricious coating having a first hydrogel layer and a second hydrophobic top coating which prevents the hydrogel coating from prematurely absorbing too much moisture. The hydrophobic top coating comprises at least one hydrophilic surfactant which acts as a carrier to facilitate removal of the hydrophobic top coating upon entry into an aqueous environment.

U.S. Pat. No. 6,261,630 disclosed a coating gradient for lubricious coatings on balloon catheters. This invention relates to a dilatation balloon formed from an extruded tubular preform by blowing, said balloon having a body, at least one cone and at least one waist portion wherein said balloon has a lubricity coating gradient from the body portion which has the lowest coat thickness to the waist portion which has the highest coat thickness said coating applied to said extruded tubular preform prior to forming said balloon by blowing.

U.S. Pat. No. 7,015,262 disclosed hydrophilic coatings for medical implements. This invention disclosed compositions, methods, devices and kits utilizing water-based hydrophilic coating formulations on medical implements. The composition for applying a coating comprises a sulfonated polyester, water, and a surface active agent. Methods for coating a medical implement comprise providing an aqueous dispersion comprising sulfonated polyester and surface active agent, contacting the medical implement with the aqueous dispersion, and drying the medical implement. Methods for acquiring a sample of bodily fluid from a patient comprise coating a needle with a sulfonated polyester, penetrating the needle into the patient, and drawing bodily fluid through the needle.

U.S. Pat. No. 7,402,620 disclosed a lubricant coating vehicle for medical devices used to reduce the coefficient of friction of such devices upon exposure thereof to moisture. The lubricant coating vehicle allows the introduction of a pharmacological additive having a release rate that is within acceptable pharmacokinetic criteria. The release rate is adjusted by utilizing different salt forms of the additive and adjusting the concentration of a urethane pre-polymer.

U.S. Pat. No. 7,691,476 disclosed hydrophilic polymeric coatings for medical articles. The invention provides a durable, lubricious coating for a medical article that can be prepared from a first polymer that is synthetic, soluble in a polar liquid, and having first reactive groups, and a second polymer that is synthetic, hydrophilic, and that includes second reactive groups. The first reactive groups and a portion of the second reactive groups react to bond the first polymer to the second polymer. A portion of the second reactive groups remains unbonded which, upon neutralization, provide lubricious properties to the coating. In some aspects the coating is formed using a crosslinking agent having latent reactive groups.

U.S. Pat. No. 8,377,559B2 disclosed methods of applying a hydrophilic coating to a substrate, and substrates having a hydrophilic coating. This invention relates to methods of applying to a substrate a hydrophilic coating that becomes lubricious when activated with water or water vapor, and to substrates having such a hydrophilic coating.

U.S. Pat. No. 8,728,508 disclosed hydrophilic coating and a method for the preparation thereof. The invention provides a method for the preparation of a cross-linked hydrophilic coating of a hydrophilic polymer on a substrate polymer surface of a medical device, involving the use of a polymer solution comprising 1-20% by weight of a hydrophilic polymer, 0-5% by weight of additive(s), and the balance of a vehicle with plasticizing effect on the hydrophilic polymer, wherein the vehicle comprises at least one plasticizer having a solubility in water of at least 6 g/L, a boiling point above 210° C. at 760 mmHg, and Hansen δH parameter of less than 20. Furthermore, the invention provides a medical device, e.g. a catheter or guide wire, provided with such a hydrophilic coating. The invention also provides the use of specific polymer solution for the preparation of a cross-linked hydrophilic coating.

U.S. Pat. No. 8,809,411 disclosed a hydrophilic coating. The invention relates to a coating formulation for preparing a hydrophilic coating, wherein the hydrophilic coating formulation comprises a hydrophilic polymer, a supporting polymer comprising a backbone and at least 2 reactive moieties capable of undergoing polymerization reactions, a Norrish Type I photoinitiator and a Norrish Type II photoinitiator.

U.S. Pat. No. 8,888,759 disclosed a medical device with hydrophilic coating. A medical device is disclosed, comprising a substrate and a hydrophilic surface coating arranged on said substrate. The substrate has, on its surface coated with said hydrophilic surface coating, a surface texture with an arithmetical mean deviation of the surface profile (Ra) of at least 3 μm and/or a profile section height difference (Rdc (1-99%)) of at least 18 μm.

U.S. Pat. No. 9,375,517B2 disclosed a lubricious medical device coating with low particulates. Embodiments of the invention include lubricious medical device coatings. In an embodiment the invention includes a coating for a medical device including a first layer comprising polyvinylpyrrolidone derivatized with a photoreactive group; and a first cross-linking agent comprising at least two photoreactive groups; a second layer disposed on the first layer comprising polyvinylpyrrolidone derivatized with a photoreactive group; a second cross-linking agent comprising at least two photoreactive groups; and a polymer comprising polyacrylamide, the polymer derivatized with at least one photoreactive group. Other embodiments are included.

U.S. Pat. No. 10,058,635B2 disclosed surface treatment agents that enable surfaces with a chemically fixed lubricant to be produced instead of a resin coating which has drawbacks, such as that lubricity is reduced due to separation, peeling or the like of the coating during the movement within a vessel or tube; and medical devices, such as catheters, having a surface at least partially treated with such a surface treatment agent. The invention relates to a surface treatment agent for medical devices which contains a copolymer of a hydrophilic functional group-containing monomer and an epoxy group-containing monomer.

U.S. Pat. No. 10,850,009B2 disclosed a medical device with hydrophilic coating. A urinary catheter having an insertable shaft formed from a blend of an ethylene and/or propylene based polymer and water swellable material. The catheter having a hydrophilic coating disposed on the outer surface of the insertable catheter shaft.

US patent application 20110015724A1 disclosed a medical device having hydrophilic coatings. The invention relates to a medical device having a coating comprising at least one polyurethane urea, wherein the coating comprises at least one polyurethane urea terminated with a copolymer unit of polyethyloxide and polypropyloxide.

European patent 1615677B1 disclosed a coating for biomedical devices. A coating formulation for a substrate having abstractable hydrogen radicals is disclosed. The formulation includes a hydrophilic polymeric component comprising at least two polymeric species of differing molecular weights, an unsaturated hydrophilic monomer capable of free-radical polymerisation in the presence of a radical and a UV activatable compound capable of abstracting hydrogen radicals from the surface to be coated and from a polymeric specie of the hydrophilic polymeric component so as to initiate and promote the cross-linkage of the monomer to the surface and of the monomer or a propagating monomer chain to a polymeric specie of the polymeric component, and a suitable solvent to give the formulation a desired viscosity.

European patent 1667747B1 disclosed lubricious coatings for medical device. This invention relates generally to the field of synthetic polymeric coating compositions for polymeric and metal substrates, to methods of making and using the same, and to articles coated therewith.

European patent 1809345B1 disclosed a medical device having a wetted hydrophilic coating. The invention relates to a medical device having a wetted hydrophilic coating comprising: a coating composition containing a hydrophilic polymer and a wetting agent comprising water and one or more lubricant(s).

European patent 1957129B1 disclosed hydrophilic coating comprising a polyelectrolyte. This invention relates to a hydrophilic coating formulation which when cured results in a hydrophilic coating. The invention further relates to a coating system, a hydrophilic coating, a lubricious coating, use of a polyelectrolyte and a non-ionic hydrophilic polymer in a lubricious coating, an article, a medical device or component and a method of forming a hydrophilic coating on a substrate.

At present, the coating material is applied to the surface of the catheter mainly by dipping or spraying, and then the coating is cured by heat or UV. Although the coating obtained in this way has a relatively lower surface coefficient of friction, because the coating is relatively thick, the bulk material of coating itself is unstable, and it is easy to fall off during use. This is particularly a problem for intravascular catheters, for which particulates released by surface coating could cause the formation of thrombus. Bulk coating material could also block catheter's lumens. Another issue with current technology is that in most cases coating can only be applied to outside surface. It is hard to modify catheters' internal surface or catheters with complicated shapes. This is especially important for catheters working together with guiding wires or dilators, which requires good internal lubricity and smoothness as well. The hydrophilic catheters, in terms of its stability, lubricity, and processing methods, need more improvement. To solve the problems with current catheters, one object of the present invention is to provide safe, stable, lubricating, strong-adhered hydrophilic coatings on catheter's external and internal surfaces, thereby improving the safety and comfort of the use of medical catheters.

### 1. Catheter Material

Common catheters on the market are mainly made of polymer materials, including silicone rubber, polyurethane, rubber, polyetheretherketone, polyethylene, polypropylene, polyvinyl chloride, nylon, ABS, polycarbonate, etc., and the present invention can be implemented on the surface of the above materials.

### 2. The Structure of Zwitterions

A variety of zwitterionic monomers can be used to graft onto the surface of the catheter to obtain a hydrophilic coating. Zwitterions can be classified according to their skeleton structure, their anionic groups, or their cationic groups. zwitterionic polymers' skeleton structures are very diverse. The most common skeleton structure includes polyolefin, such as poly(methyl)acrylamide skeletons, poly(meth)acrylate skeletons, etc. In addition, some novel polymer skeletons with unique structures have also been applied to zwitterionic polymers, including polypeptide or polypeptide-like skeletons, polyester skeletons, polysaccharide skeletons and heteroatomic backbones. There are four main types of cationic groups of zwitterionic polymers: quaternary ammonium cations, quaternary phosphonium cations, pyridinium cations, and imidazolium cations. There are three main types of anionic groups: sulfonate anions, carboxylate anions, and phosphate anions. The two combinations between anionic and cationic groups can construct different zwitterions, of which the combination of quaternary ammonium cations and different types of anions to obtain sulfonate betaine (SB), carboxylic acid betaine (CB) and phosphorylcholine (PC) is the most widely used. In addition, a amino acids, as a class of natural zwitterion, can also be applied to the side chains of zwitterionic polymers. CB, SB, PC as the three most common zwitterions have theft own unique properties. The hydration layer of the SB group can retain a large number of water molecules, and has a certain degree of self-association behavior. The hydration layer of the CB group can extend the retention time of individual water molecules, The SB group also has the characteristics of not being affected by the pH of the solution, while the CB group has the advantages of further functional modification and easy protein fixation. PC groups are an important component of phospholipid molecules, and zwitterionic polymers containing PC groups have similar properties to phospholipid molecules and can be used as polymer materials for biomimetic membranes.

### 3. Grafting Methods

Grafting reaction is needed to chemically bond the zwitterions with catheter substrate material. In general, almost all polymerization methods can be used to graft zwitterions to polymeric catheter surface, so for the present invention, the grafting methods can be used include but not limited to: Atom transfer radical polymerization (ATRP), ring-opening metathesis polymerization (ROMP), ultraviolet (UV) free radical polymerization, heat free radical polymerization, reduction-oxidation (redox) free radical polymerization, anionic or cationic polymerization, ring-opening polymerization, nitroxide-mediated radical polymerization, reversible addition-fragmentation chain-transfer polymerization (RAFT), telluride-medicated polymerization, and acyclic diene metathesis polymerization. In a preferred embodiment, an atom transfer radical polymerization reaction or a radical polymerization reaction of ultraviolet, thermal or redox is employed.

### (1) ATRP

Atom transfer radical polymerization (ATRP) is a means of forming a carbon-carbon bond with a transition metal catalyst. In ATRP reaction, free radicals are the active species, and the atom transfer step is crucial for uniform polymer chain growth.

In an ATRP process, the number of polymer chains is determined by the number of initiators. The most often used ATRP initiators include but not limited to alkyl halides, benzylic halides, α-bromo ester, α-halogenated ketone, α-halogenated nitrile, aryl sulfonyl chloride, azodiisobutyronitrile. In present invention alkyl halides, especially chloroalkanes and bromoalkanes, are preferably used as the initiator.

ATRP usually employs a transition metal complex as the catalyst, such as Cu, Fe, Ru, Ni, and Os. In an ATRP process, the dormant species is activated by the transition metal complex to generate radicals via one electron transfer process. Simultaneously the transition metal is oxidized to higher oxidation state. This reversible process rapidly establishes an equilibrium that is predominately shifted to the side with very low radical concentrations. In present invention, copper salts, especially copper chloride and copper bromide, are preferably used as the catalyst.

### (2) UV Free Radical Polymerization

UV free radical polymerization can be initialized with UV initiator, which can be introduced into the catheter substrate by mixing, imbibing or other methods. Catheters loaded with the initiator can then been put into the solution with zwitterion monomers, and after shedding with UV light, the grafting reaction can happen on the catheter surface. Normally used UV initiators include but not limited to diphenyl(2,4,6-trirnethylbenzoyl)phosphine oxide, ethyl (2,4,6-trimethylbenzoyl) phenylphosphinate, 2-methyl-4-(methylthio)-2-morpholinopropiophenone, 2-isopropyl thioxanthone, ethyl 4-dimethylaminobenzoate, 1-hydroxycyclohexyl phenyl ketone, 2-hydroxy-2-methylpropiophenone, methyl 2-benzoylbenzoate, benzophenone and derivatives.

### (3) Heat Free Radical Polymerization

Heat free radical polymerization can be initialized by adding thermal initiators into the catheter substrate material and then heating to generate heat free radicals. Considering the possible effects on catheter substrate materials, the heating temperature is in general lower than 100° C., preferably lower than 80° C. Normally used thermal initiators include but not limited to: dicumyl peroxide, potassium peroxydisulfate, peroxyacetic acid, tert-butyl peroxyacetate, tert-butyl hydroperoxide, cumene hydroperoxide, 1,1-di(tert-butylperoxy)cyclohexane, 2,2′-azobis(isobutyronitrile), 4,4′-azobis(4-cyanovaleric acid).

### (4) Redox Free Radical Polymerization

Redox initiators can be introduced into catheter substrates, and free radicals, generated via reduction-oxidation reaction, can then initiate the polymerization process. Redox initiators in general include both oxidants and reductants. One advantage of redox polymerization is its fast initiation rate and mild polymerization conditions, which in general doesn't require the same high temperature as in the thermal free radical polymerization. Normally used oxidants include but not limited to hydrogen peroxide, persulfates, hydroperoxides, pyrosulfates, pyrophosphates, permanganates, manganese(III) salts, ceric salts, ferric salts, cyclohexanone peroxide, methyl ethyl ketone peroxide, and benzoyl peroxide. Normally used reductants include but not limited to ferrous salts, chromous salts, cupric salts, titanous salts, thiols, sulfates, and bisulfates.

### EXAMPLES

### Example 1 Hydrophilic Catheters by ATRP Method

Step 1: 100 gram of N,N-dimethylaminoethyl methacrylate was dissolved into 1000 ml of glacial acetic acid. 40 gram of ethenesulfonyl chloride was slowly added into the solution, which was then stirred at room temperature for 24 hours. The precipitate was collected, washed in anhydrous ethanol twice, and then ground into powder after drying.

Step 2: Polyurethane catheters were first treated with chlorine plasma, and then added into 100 ml of 1:1 (v:v) methanol aqueous solution, which contained of 10 mM of CuCl₂, 20 mM of N,N,N′, N′, N″-pentamethyldiethylenetriamine, and 10% (w/v) of the product from Step 1. After sealing, samples and solution were purged with nitrogen for 15 minutes and then heated to 60° C. After 3 hours, the catheters were taken out, first washed with the mixture of methanol and water, then washed with saline and water, and dried in air.

### Example 2: Hydrophilic Catheters by UV Free Radical Polymerization

Step 1:100 gram of N,N-dimethylaminoethyl methacrylate was dissolved into 1000 ml of acetonitrile. Then 80 gram of 1,4-butanesultone and 300 mg of 1,3-dinitrobenzene were slowly added to the solution, which was refluxed at room temperature for 24 hours. The precipitate was collected, washed twice in acetonitrile, and dried at room temperature.

Step 2: Silicone catheters were washed and cleaned, then immersed in 100 ml of 0.1M benzophenone in ethanol for 60 minutes. After drying in air, samples were put in 100 ml of 10% (w/v) solution of the product from Step 1 in water, which was then purged with nitrogen for 15 minutes and reacted in UV rotation reactor for 6 hours. Catheters were then taken out, rinsed with saline and water, and dried in air.

### Example 3: Hydrophilic Catheters by Heat Free Radical Polymerization

Step 1: 100 gram of N,N-dimethylarninoethyl methacrylate was dissolved into 600 ml of anhydrous acetone. 55 gram of β-propiolactone was slowly added into the solution and then reacted under nitrogen at 15° C. for 6 hours. The precipitate was collected, washed with anhydrous acetone twice, dried, and then ground into powder.

Step 2: Natural rubber catheters were first immersed into 100 ml of 1% (w/v) azobisisobutyronitrile in ethanol for 60 minutes, dried in air, and then put into 100 ml of 10% (w/v) solution of product from Step 1 and 1 mM of FeCl₂. After purging with nitrogen for 15 minutes, the solution was heated to 80 ° C. and reacted for 3 hours. Then the catheters were taken out, washed with saline and water, and dried.

### Example 4: Hydrophilic Catheters by Redox Free Radical Polymerization

Step 1: 100 gram of N,N-dimethylaminoethyl methacrylate was added into 400 ml of anhydrous acetone, and 75 gram of 1,3-propanesultone was dissolved into 100 ml of anhydrous acetone. The two solutions were slowly mixed together, stirred at room temperature for 4 hours, and left at room temperature for 7 days. Then the precipitate was collected, washed with anhydrous acetone and dried.

Step 2: PVC catheters were immersed in 100 ml of 1% (w/v) tent-butyl hydroperoxide in methanol for 60 minutes, dried in aft, and then put into 100 ml of 10% (w/v) solution of the product from Step 1 and 1 mg/ml of diammonium cerium(IV) nitrate in water. After purging with nitrogen for 15 minutes, the solution was heated to 60° C. and reacted for 3 hours. Then the catheters were taken out, washed with saline and water, and dried.

### Example 5 Physical Testing of Hydrophilic Catheters

The catheters prepared in Example 1 to 4 were cleaned and dried, and the measurement found no change in size or appearance. Catheters' surface were smooth without defects, and the labels and marks on the catheter surface were clear and complete.

According to EN 1618:1997 “Catheters other than intravascular catheters—Test methods for common properties” Appendix B or ISO 10555-1:2013 “Intravascular catheters—Sterile and single-use catheters—Part 1: General requirements” Appendix B, the physical properties of the coated catheters were tested, and the uncoated catheters with the same size were used as control samples. There was no difference in the physical tensile properties between the samples before and after coating.

### Example 6: The Measurement of Surface Friction Coefficient

To measure the surface friction coefficient of control and modified catheters, ASTM standard D1894-14 “Standard Test Method for Static and Kinetic Coefficients of Friction of Plastic Film and Sheeting” was referenced with modification. Specifically, the catheters prepared in Examples 1 to 4 were placed in parallel in a friction tester containing normal saline. The two ends of the catheter were fixed horizontally at the bottom of the container and a slider with a mass of 200 grams was placed on the catheters. The slider was pulled to determine the wet friction coefficient. The tester's measurement range was 0 to 5 N and the test accuracy was not less than 0.2%. When the mold moved at a speed of 100 mm/min, the dynamic friction coefficient was measured. It has been measured that the coefficient of friction of the catheter without a surface hydrophilic coating was between 0.5 and 1, and the friction coefficient of the hydrophilic catheter prepared according to the present invention was less than 0.05, which indicates that the friction coefficient of the hydrophilic is reduced by more than an order of magnitude.

### Example 7 The Coating on the Internal Surface

Catheters prepared in Examples 3 to 4 were cut into half and the internal surface was exposed. The internal and external surfaces were measured with an Attenuated Total Reflectance-Infrared Spectroscope (ATR-IR). The coatings on internal surface and external surfaces were confirmed by the coating material fingerprint regions.

### Example 8 The Stability of the Hydrophilic Coating on Catheters

According to Chinese GB/T 14233.1-2008 “ Test methods for infusion, transfusion, injection equipment for medical use—Part 1: Chemical analysis methods”, the catheters prepared in Example 1 to 4 were soaked in 37 degrees of purification water for 72 hours by the ratio of 0.2 g/ml and purified water was used as the control sample. 50 ml of solution was taken respectively for evaporation. Compared with the control samples\, the weight gain from the testing solutions after evaporation didn't exceed 5 mg, which proved those the hydrophilic coatings were stable without peeling-off.

### Example 9 The Stability of the Coating on Hydrophilic Catheters in Artificial Gastric and Artificial Intestinal Fluids

Artificial gastric and artificial intestinal fluids were prepared according to the Chinese Pharmacopoeia 2020 edition. Six catheters from Example 1 to 4, respectively, were soaked in 37° C. artificial gastric or artificial intestinal fluid for 30 days. After removal, catheters' coefficients of friction were tested according to the method used in Example 6. It has been found that the friction coefficients didn't change after soaking.

### Example 10 The Stability of the Coating on Hydrophilic Catheters in Artificial Urine

Six catheters prepared according to Example 1 to 4, respectively, were soaked in 37° C. artificial urine in accordance with ISO 20696:2018 “Sterile urethral catheters for single use” for 30 days. After removal, catheters' coefficients of friction were tested according to the method used in Example 6. It has been found that the friction coefficients didn't change after soaking.

### Example 11 Aging Stability of Hydrophilic Catheters

According to Chinese YY/T 0681.1-2018 “Test methods for sterile medical device package—Part 1: Test guide for accelerated aging”, catheters prepared in Example 1 to 4 were stored at 55° C. for 80 days, and then the physical properties and surface friction properties of the catheter were tested according to the methods used in Example 5 and Example 6. Their physical properties and surface friction properties of the catheters before and after the aging test were measured without difference.

### Example 12 Sterilization of Hydrophilic Catheters

The catheters prepared in Example 1 to 4 were sterilized in the ethylene oxide sterilization cabinet at 55° C., 60% humidity, 1.0 g/L ethylene oxide for four hours. After degassing, microbial testing was preformed and no microbe were detected on the surface of the catheter. catheters' coefficients of friction were tested according to the method used in Example 6. It has been found that the friction coefficients didn't change after sterilization.

### Example 13 Biocompatibility of Hydrophilic Catheters

Catheters made in Examples 1 to 4 were tested for in vitro cytotoxicity (ISO 10993-5:2009 Biological evaluation of medical devices—Part 5: Tests for in vitro cytotoxicity), irritation, and skin sensitization (ISO 10993-10:2010 Biological evaluation of medical devices—Part 10: Tests for irritation and skin sensitization). In brief, in vitro cytotoxicity is based on MTT method, according to the quantitative determination criteria of the survival rate of the cultured cells (L929 mouse fibroblasts). It has been found that 100% extract of the test samples did not have cytotoxic reactions. The irritation test was carried out by intradermal reaction test method. The results showed that the final scores of the polar and non-polar extracted liquid intradermal reactions (rabbits) of the test samples were less than 1.0 and there was no skin irritation. The skin sensitization test was based on the maximum dosage method, and the skin response level in the provocation stage of all animals (guinea pigs) was 0. So no polar and non-polar extracts of the test samples were observed to cause animal sensitization. The above test results proved that the hydrophilic catheters have good biocompatibility.

The above examples only showed preferred embodiments of the present invention. It should be noted that for those of ordinary skill in the art, without departing from the technical principles of the present invention, improvements and modifications can be made. These improvements and modifications should also be regarded as the scope of protection of the present invention.

We claim:

1. Catheters comprising surface hydrophilic coatings, wherein said catheters have been grafted with at least one zwitterionic polymer, said at least one zwitterionic polymer which forms a lubricious water film and reduces surface friction when introduced to a liquid environment.

2. The catheters of claim 1, where the cationic groups of the zwitterionic polymers are quaternary ammonium, quaternary phosphonium, pyridinium, or imidazolium.

3. The catheters of claim 1, where anion groups of the zwitterionic polymers are sulfonate, carboxylic, or phosphate.

4. The catheters of claim 1, where the zwitterionic polymer is sulfobetaine, carboxybetiane, or phosphorylcholine.

5. The catheters of claim 1, where the substrate materials include silicone rubber, polyurethane, rubber, polyetheretherketone, polyethylene, polypropylene, polyvinyl chloride, nylon, ABS, polycarbonate.

6. The catheters of claim 1, where the method of surface grafting is atomic transfer, ultraviolet, thermal, or redox free radical polymerization.
